(19) United States
(12) Patent Application Publication
(10) Pub. No.: US 2021/0380529 A1
(43) Pub. Date: Dec. 9, 2021

(54) PREPARATION OF METFORMIN HYDROCHLORIDE FREE FROM GENOTOXIC IMPURITY

(71) Applicants: EXEMED PHARMACEUTICALS, Gujarat (IN); CADILA HEALTHCARE LIMITED, Gujarat (IN)

(72) Inventors: Pranav Dushyantbhai PATEL, Gujarat (IN); Mayurkumar Lalitbhai GURJAR, Gujarat (IN); Pravinchandra Jayantilal VANKAWALA, Gujarat (IN); Amit Chimanbhai PATEL, Gujarat (IN)

(21) Appl. No.: 17/337,502

(22) Filed: Jun. 3, 2021

(30) Foreign Application Priority Data
Jun. 3, 2020 (IN) 202021023234

(51) Int. Cl. C07C 277/02; C07C 277/06

(52) U.S. Cl. CPC C07C 277/02; C07C 279/26; C07C 277/06

(57) ABSTRACT

A high purity metformin hydrochloride, having a purity equal to or greater than 99% by area percentage of LCMS and no detectable amount of N-Nitrosodimethylamine (NDMA), wherein the LCMS method for the determination of NDMA content in Metformin hydrochloride has a Limit of detection of 0.004 ppm and a Limit of quantification of 0.009 ppm.

This application claims priority to IN Patent Application No. 202021023234 filed Jun. 3, 2020, the entire contents of which are hereby incorporated by reference.

### FIELD OF THE INVENTION

The present invention relates to high purity metformin hydrochloride free from genotoxic impurities and process for its preparation. The invention also relates to a pharmaceutical composition comprising high purity metformin hydrochloride free from genotoxic impurities for the treatment of diabetes.

### BACKGROUND OF THE INVENTION

The following discussion of the prior art is intended to present the invention in an appropriate technical context and allow its significance to be properly appreciated. Unless clearly indicated to the contrary, however, reference to any prior art in this specification should be construed as an admission that such art is widely known or forms part of common general knowledge in the field.

Metformin is the member of the biguanide class of an oral anti-hyperglycemic and available in various salt forms, e.g. hydrochloride. Metformin is used in the management of type 2 diabetes mellitus. It is an anti-hyperglycemic agent which improves glucose tolerance in patients with type 2 diabetes, lowering both basal and postprandial plasma glucose. Chemically, Metformin hydrochloride is 1-carbam-imidamido-N,N-dimethylmethanimidamide hydrochloride with the following structure:

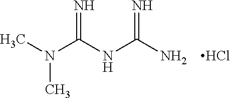

Metformin hydrochloride is widely used alone or in combination with other medicines to treat type 2 diabetes. It is usually the first-line treatment, and it works by reducing the production of glucose in the body and reducing its absorption from the gut. Metformin hydrochloride is used in various combinations with DPP IV inhibitors as add-on therapy for example sitagliptin, vildagliptin, saxagliptin, alogliptin and linagliptin.

Metformin hydrochloride is also used in various combinations with SGLT2 inhibitors as add-on therapy for example dapagliflozin, canagliflozin, empagliflozin and ertugliflozin. It's also used in combination with various drugs for example glipizide, glyburide, pioglitazone, rosiglitazone, repaglinide, glimepiride and many others.

Several processes have been reported for the preparation of metformin hydrochloride for example, in FR 2322860 B1, CN 100391939 C, IN189077 A1, and WO 2005/033089.

The Health Science Authority (HSA) of Singapore have reported for the presence of N-Nitrosodimethylamine (NDMA) in atleast three lots of metformin hydrochloride tablets brands in market.

The processes reported in the prior arts for the preparation of metformin hydrochloride does not provide teachings to control NDMA which is reported to be a potential genotoxic impurity, in the finished Active Pharmaceutical Ingredient (API). NDMA, is also known as dimethyl nitrosamine (DMN) is a semi volatile organic chemical that forms in both industrial and natural processes. It is member of N-nitrosamines, a family of potent carcinogens. It is represented by the chemical structure as depicted in Formula (II), as herein below.

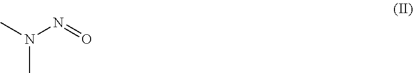

NDMA is found at low levels in numerous items of human consumption, including cured meat, fish, beer, tobacco smoke. NDMA has been classified by IARC (The International Agency for Research on Cancer) as probably carcinogenic to humans.

NDMA can occur in drinking-water through the degradation of dimethyl hydrazine as well as from several other industrial processes. NDMA is also an unintended byproduct of the chlorination of wastewater and drinking water at treatment plants that use chloramines for disinfection. In water, NDMA is completely miscible and is not expected to sorb onto solid particles or sediment. As a result of exposure to sunlight or by natural biological processes, NDMA may break down in water.

The WHO guideline on drinking water quality indicates that the most common process for NDMA removal from drinking water is UV irradiation. A concentration below 0.005 μg/L should be achievable by UV irradiation provided that the water is not grossly contaminated. Based upon the literature reference available, NDMA is not removable by air stripping, activated carbon adsorption, reverse osmosis or biodegradation.

NDMA is a common contaminant found in water and foods including cured and grilled meats, dairy products and vegetables. Everyone is exposed to some level of NDMA. The FDA and the international scientific community do not expect it to cause harm when ingested at low levels. The acceptable daily intake limit for NDMA in the U.S. is 96 nanograms. Genotoxic substances such as NDMA may increase the risk of cancer if people are exposed to them above acceptable levels and over long periods of time.

NDMA is formed by the reaction of nitrous acid with dimethylamine, as herein below.

HONO+(CH₃)₂NH→(CH₃)₂NNO+H₂O

NDMA is postulated to be present in drug substances prepared using N,N-dimethylformamide (DMF) as solvent. Experts suggests that during the breakdown of DMF, small amounts of dimethylamine may also be formed, which in the presence of nitrous acid, can become a potential source of NDMA.

Prior art processes do not disclose removal of NDMA impurity and other genotoxic impurities in metformin hydrochloride and process for the preparation of high purity metformin hydrochloride free from NDMA.

The present invention provides high purity metformin hydrochloride free from NDMA and pharmaceutical composition comprising metformin free from NDMA for the treatment of diabetes.

### SUMMARY OF THE INVENTION

In one general aspect, there is provided high purity metformin hydrochloride free from N-Nitrosodimethylamine (NDMA).

In one general aspect, there is provided high purity metformin hydrochloride, having a purity equal to or greater than 99% by area percentage of LCMS and no detectable amount of NDMA, wherein, the LCMS method for the determination of NDMA content in Metformin hydrochloride has a Limit of detection of 0.004 ppm and a Limit of quantification of 0.009 ppm.

In another general aspect, there is provided high purity metformin hydrochloride, having a purity equal to or greater than 99% by area percentage of LCMS and NDMA in an amount less than threshold of toxicological concern limit (TTC) 0.035 ppm.

In another general aspect, there is provided a process for the preparation of high purity metformin hydrochloride, the process comprising:
(a) reacting dicyanodiamide of Formula (III) with dimethylamine hydrochloride of Formula (IV) in one or more hydrocarbon solvents;

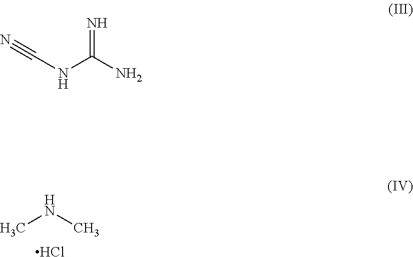

(b) extracting reaction mass with water to obtain a solution;
(c) treating the solution with carbon;
(d) removing the water to obtain a residue;
(e) treating the residue with one or more C₁-C₄ alcohols to obtain a slurry;
(f) pulverizing the slurry; and
(g) drying at 60-85° C. to obtain the metformin hydrochloride having no detectable amount of NDMA.

In another general aspect, there is provided a process for the preparation of high purity metformin hydrochloride comprising treating metformin hydrochloride with highly active carbon, optionally in the presence of one or more solvents.

In another general aspect, there is provided a process for the preparation of high purity metformin hydrochloride free from NDMA comprising treating metformin hydrochloride with highly active carbon, optionally in the presence of one or more solvents and drying the product at 60-85° C.

In another general aspect, there is provided a pharmaceutical composition comprising metformin hydrochloride having a purity equal to or greater than 99% by area percentage of LCMS and NDMA in an amount less than TTC 0.035 ppm, prepared by the process as mentioned herein above.

In another general aspect, there is provided a pharmaceutical composition comprising high purity metformin hydrochloride having a purity equal to or greater than 99% by area percentage of HPLC and NDMA in an amount, less than TTC 0.035 ppm.

In another general aspect, there is provided a pharmaceutical composition comprising high purity metformin hydrochloride having a purity equal to or greater than 99% by area percentage of HPLC and NDMA in an amount, less than TTC 0.035 ppm and one or more pharmaceutically acceptable carriers, excipients or diluents.

In another general aspect, there is provided a pharmaceutical composition comprising metformin hydrochloride having a purity equal to or greater than 99% by area percentage of LCMS and NDMA in an amount less TTC 0.035 ppm, alone or in combination with at least another therapeutic agent in the presence of a pharmaceutically acceptable carrier.

In another general aspect, there is provided a pharmaceutical compositions comprising high purity metformin hydrochloride free from NDMA for the treatment of diabetes.

In another general aspect, there is provided a pharmaceutical composition comprising high purity metformin hydrochloride free from NDMA and one or more DPP IV inhibitors.

In another general aspect, there is provided a pharmaceutical composition comprising high purity metformin hydrochloride free from NDMA and one or more SGLT2 inhibitors.

In another general aspect, there is provided a method for treating diabetes in patients comprising administering a therapeutically effective amount of a pharmaceutical composition comprising metformin hydrochloride having a purity equal to or greater than 99% by area percentage of HPLC and NDMA in an amount, less than TTC 0.035 ppm, alone or in combination with at least another therapeutic agent in the presence of a pharmaceutically acceptable carrier.

### DETAILED DESCRIPTION OF THE INVENTION

As used herein the term “obtaining” may include filtration, filtration under vacuum, centrifugation, and decantation for isolation of the product. The product may be preceded for further steps with or without isolation and with or without drying in case of the product was isolated.

The term “treating” means mixing, stirring, slurring, keeping that at any temperature or conditions.

The term “cooling” herein means the reaction mixture is cooled at temperature from about 0° C. to about 35° C., In particular, from about 5° C. to about 30° C., More particularly, from about 5° C. to about 25° C., after the reaction mixture is being heated.

The term “free from NDMA” herein means metformin hydrochloride having NDMA less than TTC 0.035 ppm by area percentage of LCMS. In particular, less than about 0.010 ppm by area percentage of LCMS, and less than about 0.004 ppm. More particular, not in detectable amount by area percentage of LCMS.

### Abbreviations:

### LOQ: Limit of Quantification

### LOD: Limit of Detection

### HPLC: High Performance Liquid Chromatography

### API: Active Pharmaceutical Ingredient

### Hrs: Hours

### MRM: Multiple Reaction Monitoring

### APCI: Atmospheric Pressure Chemical Ionization

### LCMS: Liquid Chromatography Mass Spectrometry

### ppm: Parts Per Million

In general, the solvents may be removed from the reaction mixture in order to obtain solid or precipitate. The solvents may be removed by one or more of filtration, filtration under vacuum, centrifugation, decantation, distillation and distillation under vacuum.

The product(s) obtained may further be converted to any other physical forms thereof which includes but not specifically limited to salt(s), solvate(s), hydrate(s), co-crystal(s) and solid dispersion(s) in either crystalline or amorphous forms.

In one general aspect, there is provided high purity metformin hydrochloride free from NDMA.

In another general aspect, there is provided a high purity metformin hydrochloride, having a purity equal to or greater than 99% by area percentage of LCMS and no detectable amount of NDMA, wherein the LCMS method for the determination of NDMA content in Metformin hydrochloride has a Limit of detection of 0.004 ppm and a Limit of quantification of 0.009 ppm.

In another general aspect, there is provided high purity metformin hydrochloride, having a purity equal to or greater than 99% by area percentage of LCMS and NDMA in an amount less than 0.035 ppm.

In another general aspect, there is provided a process for the preparation of high purity metformin hydrochloride, the process comprising:
(a) reacting dicyanodiamide of Formula (III) with dimethylamine hydrochloride of Formula (IV) in one or more hydrocarbon solvents;

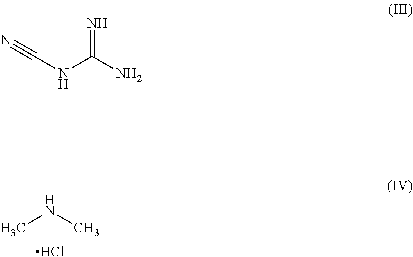

(b) extracting reaction mass with water to obtain a solution;
(c) treating the solution with carbon;
(d) removing the water to obtain a residue;
(e) treating the residue with one or more C₁-C₄ alcohols to obtain a slurry;
(f) pulverizing the slurry; and
(g) drying at 60-85° C. to obtain the metformin hydrochloride having no detectable amount of NDMA.

In general, the reaction of dicyanodiamide of Formula (III) with dimethylamine hydrochloride of Formula (IV) may be carried out in one or more hydrocarbon solvents.

The hydrocarbon solvent may be selected from one or more of toluene, xylene, ethylbenzene, cyclohexane, hexane, cyclopentane, pentane, and heptane. In particular, toluene and xylene may be used. The reaction mixture may be heated at a temperature from about 50° C. to about 150° C.

After the completion of the reaction, the reaction mass may be extracted with water at an ambient temperature to reflux temperature, for example, at about 80° C. to about 95° C. The solution may be purged with nitrogen gas for about 15 minutes to one hour.

The solution can be treated with highly active carbon to remove colored and other suspended matters and impurities.

In general, the carbon at step (c) is highly active carbon having a surface area between 1200 to 1800 m²/gm and pore volume in the range of 0.5 to 1.8 cc/gm.

The highly active carbon used according to the invention is having pore volume in the range of 0.5 to 1.8 cc/gm, preferably 0.55-1.2 cc/gm and surface area between 1200 to 1800 m²/gm, preferably 1400-1600 m²/gm.

The highly active carbon is selected from one or more of PF457, 31 AL, 31 HW, Pentacarb, New carb (N) and 21 HW or equivalents having pore volume in the range of 0.5 to 1.8 cc/gm.

The following are the grades of activated carbon which effectively removes the traces level of NDMA present in drug substance during crystallization

| Grade No. | Pore Volume | |
|---|---|---|
| PF457 | 1.2 | cc/gm |
| 31 AL | 0.52 | cc/gm |
| 31 HW | 0.62 | cc/gm |
| 21 HW | 0.55 | cc/gm |

The water may be removed from the solution by a technique which includes, for example, distillation, distillation under vacuum, and evaporation.

The residue obtained may be treated with one or more of C₁-C₄ alcohols to obtain slurry.

The C₁-C₄ alcohols at step (e) comprises one or more of methanol, ethanol, propanol, isopropanol, butanol and isobutanol.

The drying at step (g) comprises drying at 60-85° C. under vacuum (Not less than 700 mm Hg) to obtain the metformin hydrochloride having no detectable amount of NDMA.

Certain manufacturing conditions using the solvent N, N-Dimethylformamide (DMF), gave rise to this class of impurities in drug substance. NDMA is a carcinogenic risk substance and for which the acceptable intake (i.e. permissible daily exposures—PDE) is 0.0959 μg/day. Maximum daily dose of Metformin HCl is 2.55 g./day

Concentration (μg/g)=PDE (μg/day)/Daily amount of drug product (g./day)=0.0959/2.55=0.03761 in ppm≈0.038 in ppm

TTC=Threshold of Toxicological Concern (TTC)

NDMA forms by the reaction of nitrous acid with dimethylamine:

HONO+(CH₃)₂NH→(CH₃)₂NNO+H₂O

NDMA can form by following routes also:

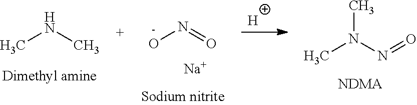

### Or

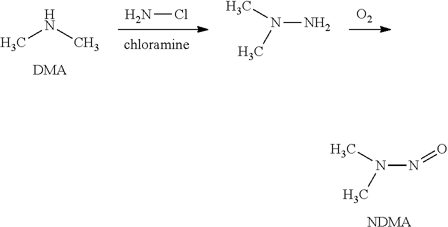

The present invention does not involve N,N-Dimethylformamide as a solvent and sodium nitrite during the process. Preparation of metformin hydrochloride in the present invention involves reaction of cyano guanidine with dimethylamine hydrochloride. Other materials used in process are xylene, methanol, activated carbon and water. As there is no scope of generation of the NDMA and other nitrosamines although dimethylamine is used. Therefore, there is no possibility of NDMA and other Nitrosamines.

The starting materials for the preparation of metformin are prepared as per the procedure described herein below.

### Preparation of Compound (III):

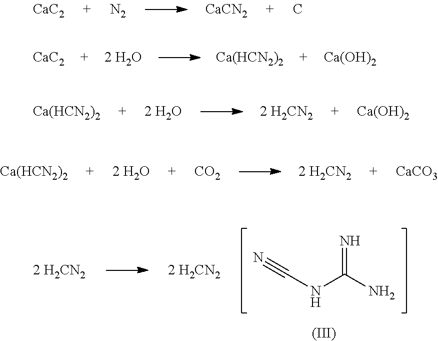

### Preparation of Compound (IV):

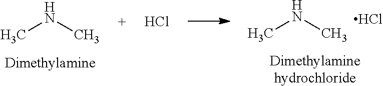

As during the synthesis of metformin hydrochloride and starting compounds-III and IV, neither sodium nitrite, nor dimethyl formamide is used in the process, therefore NDMA and other Nitrosamines are not possible in the process.

Usage of water, after analysing for NDMA before its use in the process for preparation of metformin hydrochloride:

As per WHO Guidelines, NDMA and Nitrite were analysed in water to be used for the synthesis of metformin hydrochloride.

| Test | Guideline | Result (ppm) |
|---|---|---|
| Nitrite | Max 3.0 | Not Detected (<0.01 ppm) |
| NDMA | Max 0.1 | Not Detected (<0.01 ppm) |

In another general aspect, there is provided a pharmaceutical compositions comprising metformin hydrochloride free from NDMA for the treatment of diabetes.

In another general aspect, there is provided a pharmaceutical composition comprising metformin hydrochloride having a purity equal to or greater than 99% by area percentage of LCMS and NDMA in an amount less than 0.035 ppm.

In another general aspect, there is provided a pharmaceutical composition comprising metformin hydrochloride having a purity equal to or greater than 99% by area percentage of LCMS and NDMA in an amount less than 0.035 ppm and one or more pharmaceutically acceptable carriers, excipients or diluents.

The determination of NDMA content in Metformin Hydrochloride API by LCMS:

### Experimental:

In general, the following LCMS methods for the determination of NDMA in metformin may be used.

LCMS Chromatograph Mass Spectrometry Approach:

NDMA from Synzeal batch No. SRL-255-111 having purity of 99.96% was used as standard. The method is highly sensitive to detected NDMA in metformin upto the level of 0.004 ppm with respect to metformin.

Chromatographic condition:
Equipment: HPLC System 1260 or equivalent

### Column: ACE 3 PFP C18

### Detector: Mass

Flow rate: 0.7 mL/min

### Injection Volume: 50 μL

Column temperature: 30° C.
Run time: 10 mins

### Mass Parameters:

Equipment: Q trap 4500
Scan type: MRM

### Polarity: Positive

Curtain gas (N₂): 30 mL/min

### Temperature: 400° C.

Dwell time: 200 msec

The following are the results of the batches tested using LCMS method for the determination of NDMA content in metformin.

| Batch No. | Results of NDMA in ppm |
|---|---|
| MFH2019001 | Not Detected |
| MFH2019002 | Not Detected |
| MFH2019003 | Not Detected |
| MFH2019004 | Not Detected |

In another general aspect, there is provided a pharmaceutical composition comprising high purity metformin hydrochloride having a purity equal to or greater than 99% by area percentage of HPLC and NDMA in an amount, less than TTC 0.035 ppm.

The Impact of carbon treatment and drying at different temperature is as shown in below table:

### 1. Limit of NDMA=0.035 ppm

2. Limit of detection=0.004 ppm
3. Limit of quantification=0.009 ppm

| | Carbon | | | NDMA |
|---|---|---|---|---|
| Batch No. | grade | Sample details | | Imp. ppm |
| MFH210661AFP | — | As such | As such aq. | 0.833 |
| | | aq. Mass | Mass | |
| MFH210661AFP | — | — | Before carbon | 0.295 |
| | | | treatment aq. | |
| | | | Mass | |
| MFH210661AFP | — | — | After carbon | 0.441 |
| | | | treatment aq. | |
| | | | Mass | |
| | Carbon | | Drying | NDMA |
| Batch No. | grade | Sample | temperature | Impurity |
| RD/MF/0011/20/022 | Pentacarb | Sample-01 | 60-65° C. | Not |
| | | | | Detected |
| | | Sample-02 | 70-75° C. | Not |
| | | | | Detected |
| | | Sample-03 | 80-85° C. | Not |
| | | | | Detected |
| RD/MF/0011/20/023 | Exemed | Sample-01 | 60-65° C. | 0.004 |
| | carbon | Sample-02 | 70-75° C. | Not |
| | | | | Detected |
| | | Sample-03 | 80-85° C. | Not |
| | | | | Detected |
| RD/MF/0011/20/024 | PF 457 | Sample-01 | 60-65° C. | 0.004 |
| | | Sample-02 | 70-75° C. | Not |
| | | | | Detected |
| | | Sample-03 | 80-85° C. | Not |
| | | | | Detected |
| RD/MF/0011/20/025 | New carb | Sample-01 | 60-65° C. | Not |
| | (N) | | | Detected |
| | | Sample-02 | 70-75° C. | Not |
| | | | | Detected |
| | | Sample-03 | 80-85° C. | Not |
| | | | | Detected |

In another general aspect, there is provided a pharmaceutical composition comprising metformin hydrochloride having a purity equal to or greater than 99% by area percentage of LCMS and NDMA in an amount less than TTC 0.035 ppm, prepared by the process as mentioned herein above.

In another general aspect, there is provided a pharmaceutical composition comprising metformin hydrochloride having a purity equal to or greater than 99% by area percentage of LCMS and NDMA in an amount, less than TTC 0.035 ppm and one or more pharmaceutically acceptable carriers, excipients or diluents.

In another general aspect, there is provided a pharmaceutical composition comprising metformin hydrochloride having a purity equal to or greater than 99% by area percentage of LCMS and NDMA in an amount, less than TTC 0.035 ppm alone or in combination with at least another therapeutic agent in the presence of a pharmaceutically acceptable carrier.

The therapeutic agent comprises one or more of DPP IV inhibitors, SGLT2 inhibitors or glipizide, glyburide, repaglinide pioglitazone, rosiglitazone and glimepiride.

In another general aspect, there is provided a pharmaceutical composition comprising metformin hydrochloride having a purity equal to or greater than 99% by area percentage of LCMS and NDMA in an amount, less than TTC 0.035 ppm and one or more SGLT2 inhibitors or one or more DPP IV inhibitors.

In another general aspect, there is provided a method for treating diabetes in patients comprising administering a therapeutically effective amount of a pharmaceutical composition comprising metformin hydrochloride having a purity equal to or greater than 99% by area percentage of HPLC and NDMA in an amount, less than TTC 0.035 ppm, alone or in combination with at least another therapeutic agent in the presence of a pharmaceutically acceptable carrier.

In general, the pharmaceutically acceptable carriers may be used for example lactose, sucrose, mannitol, sorbitol; cornstarch, tapioca starch and potato starch; cellulose, sodium carboxymethyl cellulose, ethyl cellulose and methyl cellulose, calcium phosphates, dicalcium phosphate, tricalcium phosphate, sodium sulfate, calcium sulfate, polyvinyl pyrrolidone, polyvinyl alcohol, stearic acid, magnesium stearate, calcium stearate, stearic acid, peanut oil, cottonseed oil, sesame oil, olive oil, corn oil, ethylene glycol betacyclodextrin, fatty alcohols, and hydrolyzed cereal solids, as well as other non-toxic compatible fillers, binders, disintegrants, buffers, preservatives, antioxidants, lubricants, flavoring agents which are commonly used in formulations.

The examples are set forth to aid in understanding the invention but are not intended to, and should not be construed to limit its scope in any way. The examples do not include detailed descriptions of conventional methods. Such methods are well known to those of ordinary skill in the art and are described in numerous publications.

### EXAMPLES

### Example-1: Preparation of Metformin Hydrochloride Free from NDMA Impurity Using PF457 Grade Carbon

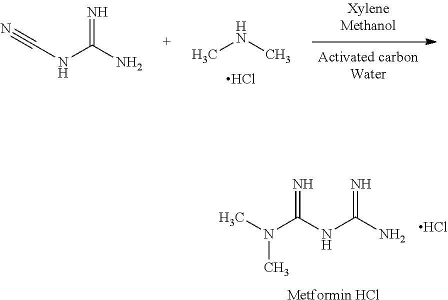

Xylene (400 mL) and dicyanodiamide (100 g) were taken in a round-bottom flask. The reaction mixture was heated at 80° C. Dimethylamine hydrochloride (117 g) was added portion-wise within 2 hours. The reaction mass was stirred for 3 hours. The reaction mixture was further heated to 100° to 105° C. followed by heating to 140° C. The reaction mass was stirred for 4 hours and cooled to 95° C. The reaction mass was treated with water (200 mL) and layers were separated. The organic layer was again extracted with water (50 mL). The combined aqueous layer was treated with carbon PF457 grade (3 g) and stirred for 20 minutes. The reaction mass was filtered through a hyflo bed and washed with water (50 mL). The filtrate was taken in a round-bottom flask at 50° C. and N₂ gas was purged for 30 minutes. The filtrate was distilled to remove water completely under vacuum at 65° C. The residue thus obtained was treated with methanol (110 mL) at 40° C. to 45° C. and cooled to 20° C. to 25° C. The product was filtered and washed with chilled methanol (50 mL). The wet-cake thus obtained was treated with water at 50° C. along with N₂ gas purging for 30 minutes. The solution was distilled to remove water completely under vacuum at 65° C. The residue thus obtained was treated with methanol (80 mL) at 40° C. to 45° C. to prepare the slurry. The slurry was pulverized under high-speed grinder for wet grinding for 25 minutes. The reaction mass was filtered and dried. The wet-cake was washed with chilled methanol (30 mL). The product was dried at 65° C. to 70° C. to obtain 160 g metformin hydrochloride having NDMA content—Not detected.

### Example-2: Preparation of Metformin Hydrochloride Free from NDMA Impurity Using 31HW Grade Carbon

Toluene (500 mL) and dicyanodiamide (100 g) were taken in a round-bottom flask at 25° C. to 35° C. The reaction mixture was heated at 80° C. Dimethylamine hydrochloride (117 g) was added portion-wise within 2 hours. The reaction mass was stirred for 3 hours. The reaction mixture was further heated to 100° to 105° C. followed by heating to reflux temperature. The reaction mass was stirred for 4 hours and cooled to 95° C. The reaction mass was treated with water (200 mL) and layers were separated. The organic layer was extracted with water (50 mL). The combined aqueous layer was treated with carbon 31HW (3 g) and stirred for 20 minutes. The reaction mass was filtered through hyflo bed and washed with water (50 mL). The filtrate was taken in a round-bottom flask at 50° C. and N₂ gas was purged for 30 minutes. The filtrate was distilled to remove water completely under vacuum at 65° C. The residue thus obtained was treated with methanol (110 mL) at 40° C. to 45° C. and cooled to 20° C. to 25° C. The product was filtered and washed with chilled methanol (50 mL). The wet-cake thus obtained was treated with water at 50° C. along with N₂ gas purging for 30 minutes. The solution was distilled to remove water completely under vacuum at 65° C. The residue thus obtained was treated with methanol (80 mL) at 40° C. to 45° C. to prepare the slurry. The slurry was pulverized under high-speed grinder for wet grinding for 25 minutes. The reaction mass was filtered and dried. The wet-cake was washed with chilled methanol (30 mL). The product was dried at 65° C. to 70° C. to obtain 155 g metformin hydrochloride having NDMA content—Not detected.

### Example-3: Preparation of Metformin Hydrochloride Free from NDMA Impurity Using Pentacarb Grade Carbon

After the reaction of Dicyanodiamide with Dimethylamine hydrochloride as explained in example 1 and 2, the product was extracted in aqueous layer. The aqueous layer (175 mL) was treated with Pentacarb grade carbon (0.1 g) and stirred at 90-100° C. for 30 minutes. The reaction mass was filtered through a hyflo bed and washed with water (25 mL). The filtrate was taken in a round-bottom flask at 50° C. and N₂ gas was purged for 30 minutes. The filtrate was distilled to remove water completely under vacuum at 65° C. The residue thus obtained was treated with methanol (55 mL) at 40-45° C. and cooled to 25-35° C. The product was filtered and washed with chilled methanol (50 mL). The wet-cake thus obtained was treated with methanol (45 mL) at 25-35° C. to prepare the slurry. The reaction mass was filtered and suck dried. The wet-cake was washed with methanol (15 mL). The product was dried under vacuum (Not less than 700 mm Hg) at temperature 80-85° C. to obtain metformin hydrochloride having NDMA content—Not detected (below 0.004 ppm).

### Example-4: Preparation of Metformin Hydrochloride Free from NDMA Impurity Using PF 457 Grade Carbon

After the reaction of Dicyanodiamide with Dimethylamine hydrochloride as explained in example 1 and 2, the product was extracted in aqueous layer. The aqueous layer (175 mL) was treated with PF 457 grade carbon (0.1 g) and stirred at 90-100° C. for 30 minutes. The reaction mass was filtered through a hyflo bed and washed with water (25 mL). The filtrate was taken in a round-bottom flask at 50° C. and N₂ gas was purged for 30 minutes. The filtrate was distilled to remove water completely under vacuum at 65° C. The residue thus obtained was treated with methanol (55 mL) at 40-45° C. and cooled to 25-35° C. The product was filtered and washed with chilled methanol (50 mL). The wet-cake thus obtained was treated with methanol (45 mL) at 25-35° C. to prepare the slurry. The reaction mass was filtered and suck dried. The wet-cake was washed with methanol (15 mL). The product was dried under vacuum (Not less than 700 mm Hg) at temperature 70-75° C. to obtain metformin hydrochloride having NDMA content—Not detected (below 0.004 ppm).

### Example-5: Preparation of Metformin Hydrochloride Free from NDMA Impurity Using New Carb (N) Grade Carbon

After the reaction of Dicyanodiamide with Dimethylamine hydrochloride as explained in example 1 and 2, the product was extracted in aqueous layer. The aqueous layer (175 mL) was treated with new carb (N) grade carbon (0.1 g) and stirred at 90−100° C. for 30 minutes. The reaction mass was filtered through a hyflo bed and washed with water (25 mL). The filtrate was taken in a round-bottom flask at 50° C. and N₂ gas was purged for 30 minutes. The filtrate was distilled to remove water completely under vacuum at 65° C. The residue thus obtained was treated with methanol (55 mL) at 40-45° C. and cooled to 25-35° C. The product was filtered and washed with chilled methanol (50 mL). The wet-cake thus obtained was treated with methanol (45 mL) at 25-35° C. to prepare the slurry. The reaction mass was filtered and suck dried. The wet-cake was washed with methanol (15 mL). The product was dried under vacuum (Not less than 700 mm Hg) at temperature 80-85° C. to obtain metformin hydrochloride having NDMA content—Not detected (below 0.004 ppm).

While the present invention has been described in terms of a few specific embodiments, modifications and equivalents thereof, in light of teaching and disclosure of the present invention that are apparent of the skilled artisan, are to be construed as included within the scope of the invention.

We claim:

1. A high purity metformin hydrochloride, having a purity equal to or greater than 99% by area percentage of LCMS and no detectable amount of N-Nitrosodimethylamine (NDMA), wherein the LCMS method for the determination of NDMA content in Metformin hydrochloride has a Limit of detection of 0.004 ppm and a Limit of quantification of 0.009 ppm.

2. The high purity metformin hydrochloride according to claim 1, having a purity equal to or greater than 99% by area percentage of LCMS and NDMA in an amount less than 0.035 ppm.

3. The high purity metformin hydrochloride according to claim 1 prepared by a process comprising:
(h) reacting dicyanodiamide of Formula (III) with dimethylamine hydrochloride of Formula (IV) in one or more hydrocarbon solvents; 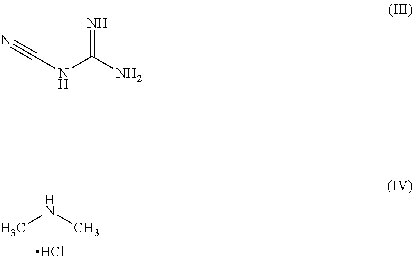
(i) extracting reaction mass with water to obtain a solution;
(j) treating the solution with carbon;
(k) removing the water to obtain a residue;
(l) treating the residue with one or more C₁-C₄ alcohols to obtain a slurry;
(m) pulverizing the slurry; and
(n) drying at 60-85° C. to obtain the metformin hydrochloride having no detectable amount of NDMA.

4. The process according to claim 3, wherein the hydrocarbon at step (a) comprises one or more of toluene, xylene, ethylbenzene, cyclohexane, hexane, cyclopentane, pentane, and heptane.

5. The process according to claim 3, wherein the carbon at step (c) is highly active carbon having a surface area between 1200 to 1800 m²/gm and pore volume in the range of 0.5 to 1.8 cc/gm.

6. The process according to claim 3, wherein the C₁-C₄ alcohols at step (e) comprises one or more of methanol, ethanol, propanol, isopropanol, butanol and isobutanol.

7. The process according to claim 5, wherein the highly active carbon is selected from one or more of PF457, 31 AL, 31 HW, Pentacarb, New carb (N) and 21 HW or equivalents having pore volume in the range of 0.5 to 1.8 cc/gm.

8. The process according to claim 3, wherein the drying at step (g) comprises drying at 60-85° C. under vacuum (Not less than 700 mm Hg) to obtain the metformin hydrochloride having no detectable amount of NDMA.

9. A pharmaceutical composition comprising metformin hydrochloride having a purity equal to or greater than 99% by area percentage of LCMS and NDMA in an amount less than threshold of toxicological concern limit (TTC) 0.035 ppm, prepared by the process according to claim 3.

10. A pharmaceutical composition comprising metformin hydrochloride having a purity equal to or greater than 99% by area percentage of LCMS and NDMA in an amount less than TTC 0.035 ppm and one or more pharmaceutically acceptable carriers, excipients or diluents.

11. A pharmaceutical composition comprising metformin hydrochloride having a purity equal to or greater than 99% by area percentage of LCMS and NDMA in an amount less than TTC 0.035 ppm, alone or in combination with at least another therapeutic agent in the presence of a pharmaceutically acceptable carrier.

12. The pharmaceutical composition according to claim 11, wherein the therapeutic agent comprises one or more of DPP IV inhibitors, SGLT2 inhibitors or glipizide, glyburide, repaglinide pioglitazone, rosiglitazone and glimepiride.

13. A method for treating diabetes in patients comprising administering a therapeutically effective amount of a pharmaceutical composition comprising metformin hydrochloride having a purity equal to or greater than 99% by area percentage of HPLC and NDMA in an amount less than TTC 0.035 ppm, alone or in combination with at least another therapeutic agent in the presence of a pharmaceutically acceptable carrier.
